(19) United States
(12) Patent Application Publication
(10) Pub. No.: US 2006/0271075 A1
(43) Pub. Date: Nov. 30, 2006

(54) Double loop gastric bypass procedure

(76) Inventors: Federico Bilotti, Aprilia (IT); Michele D'Arcangelo, Roma (IT); Alessandro Pastorelli, Rome (IT)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/437,753

(22) Filed: May 18, 2006

(30) Foreign Application Priority Data
May 27, 2005 (IT) MI2005A001004

(51) Int. Cl. A61B 17/08

(52) U.S. Cl. 606/153

(57) ABSTRACT

An instrumentation for carrying out a gastrointestinal bypass comprises means for bringing close together a first tissue portion (12) of the intestine and second tissue portion (14) of the stomach by realising a first intestine loop (A), means for creating a gastroenteroanastomosis (16) between the two close tissue portions, for example a linear stapler, maintaining or restoring the continuity of the intestinal duct after the realisation of the gastroenteroanastomosis, means for bringing close together an additional first tissue portion (18) of the intestine, arranged distally with respect to the gastroenteroanastomosis (16) with reference to the natural flow of the food along the intestinal duct, and an additional second tissue portion (20) of the intestine arranged proximally with respect to the gastroenteroanastomosis (16) with reference to the natural flow of the food along the intestinal duct, realising a second loop (B) of the intestine, wherein said additional second tissue portion (20) of the intestine arranged proximally with respect to the gastroenteroanastomosis (16) is brought close to the gastroenteroanastomosis (16), means for creating an enteroenteroanastomosis (22) between the two close tissue portions of the intestine, maintaining or restoring the continuity of the intestinal duct after the realisation of the enteroenteroanastomosis, and means for sectioning the intestine between the gastroenteroanastomosis (16) and the enteroenteroanastomosis (22).

In general terms, the present invention regards instrumentation and a method for carrying out a gastrointestinal bypass.

Such instrumentation and method are particularly adapted for being used laparoscopically or intraluminally.

Techniques are known for carrying out gastrointestinal bypasses, included the gastric bypass known as “Roux-en-Y”, following which the gastrointestinal course winds between a stomach pouch, to which a portion of the intestine is directly connected. A large part of the stomach and intestine is therefore bypassed with the creation of a gastroenteroanastomosis.

Such technique, however, has several drawbacks both in terms of high time required in execution and in terms of post-operative risks and complications.

As is known, in general both the laparoscopic and intraluminal approach considerably limit the drawbacks of the conventional surgical methodology. In particular, they permit limiting the invasiveness of the procedure, reducing the risks for the patient and shortening the post-operative course.

The currently available instrumentation does not however permit limiting the drawbacks of the prior art, and in particular of the gastrointestinal bypass techniques such as that described above. For example, the use of circular staplers, above all in the realisation of the gastroenteroanastomosis, requires large sections of the intestine, which comprise its integrity and continuity even before concluding the operation. It follows that the operations still results invasive and does not permit verifying the effectiveness and seal of the anastomosis, in particular of the enteroenteroanastomosis.

Other drawbacks of the known techniques and instrumentation are for example tied to the preliminary section of the intestine, which requires considerable experience to evaluate the correct length at which to carry out the cutting and to recognise the two sectioned flaps. A further drawback is instead due to the large area of action which substantially covers the entire patient abdomen.

The problem underlying the present invention is that of proposing instrumentation and a method for carrying out gastrointestinal bypasses which are capable of overcoming the drawbacks mentioned with reference to the prior art, thus permitting satisfying the field's growing need to limit the risks and invasiveness.

Such problem is resolved by means of instrumentation for carrying out a gastrointestinal bypass in accordance with claim 1 and by means of a method for carrying out a gastrointestinal bypass in accordance with claim 16.

Further characteristics and advantages of the instrumentation and method for carrying gastrointestinal bypasses according to the invention will be clear from the description reported below of preferred embodiments, given as indicative and not limiting, with reference to the attached figures, wherein:

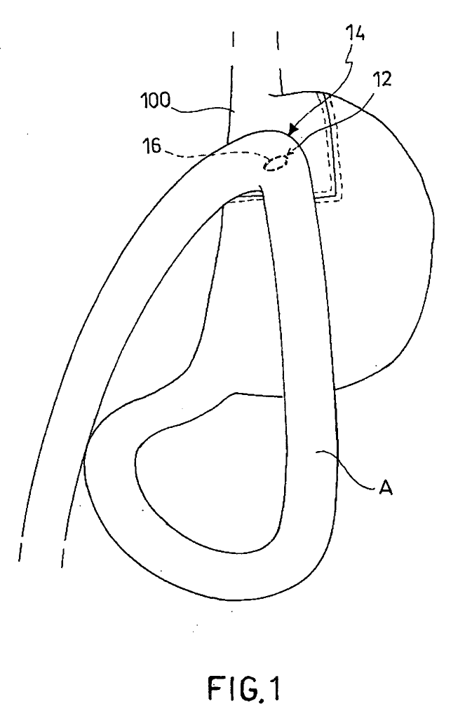

FIG. 1 illustrates a first step of the method for carrying out a gastrointestinal bypass in accordance with one aspect of the present invention;

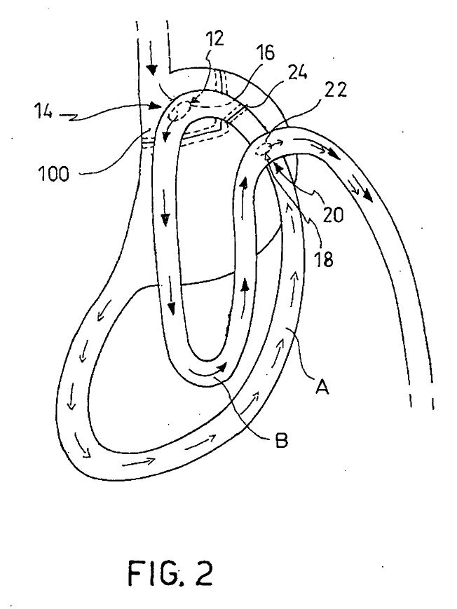

FIG. 2 illustrates a second step of the method for carrying out a gastrointestinal bypass in accordance with one aspect of the present invention;

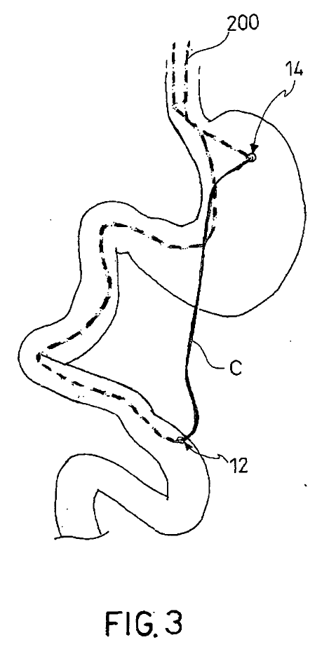

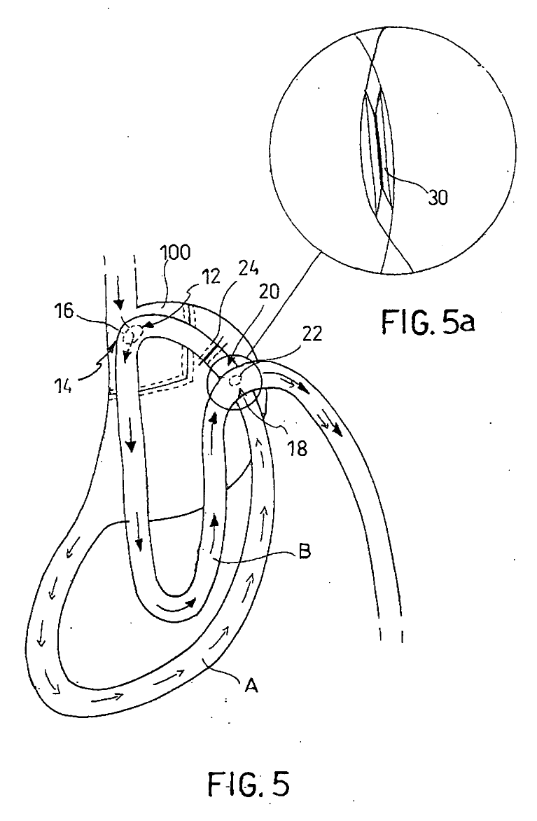

FIGS. 3-6 illustrate several steps of the method according to a further aspect of the present invention and in particular the FIG. 5a illustrates an enlarged detail of FIG. 5 according to one possible embodiment and from a different angle.

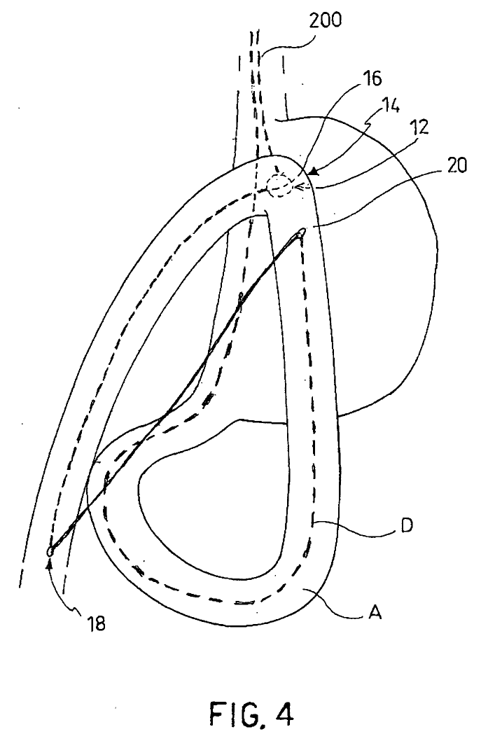

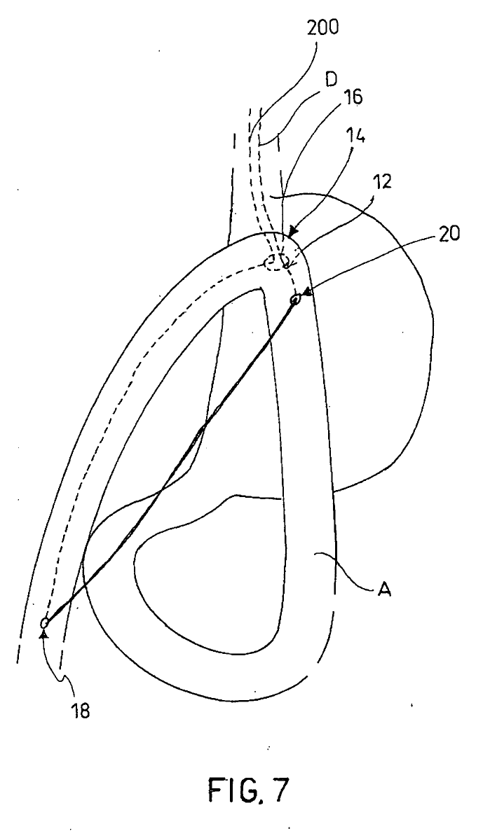

FIG. 7 illustrates a possible embodiment variation of FIG. 4.

In accordance with one possible embodiment, the present invention regards a method for carrying out gastrointestinal bypasses comprising the steps as illustrated in FIGS. 1 and 2.

Such method comprises steps of bringing close together and joining the tissues to form anastomoses adapted to maintain or restore the integrity and continuity of the intestinal duct after each anastomosis formation (whether gastroenteroanastomosis or enteroenteroanastomosis). Moreover, the gastroenteroanastomosis and the enteroenteroanastomosis are realised at close distances, balancing the contrasting needs to limit the operating zone, for example to only one upper zone of the abdomen, while keeping a wide operating and visual area.

In accordance with a possible embodiment, a first step of the method foresees realising a gastric pouch 100 to which the first portion of the intestine will be connected. Subsequently, an ansa of the intestine is selected to be united to the gastric pouch. The choice is operated by means of measuring the available length and possibly verifying that tensions or distortions are not generated.

A first tissue portion 12 of the intestine, corresponding with the chosen ansa, is therefore brought close to a second tissue portion 14 of the stomach at the gastric pouch 100. A first loop A of the intestine is thus realised which extends between the stomach and the first tissue portion 12 of the intestine.

The two tissue portions are slightly incised, forming an enterotomy and a gastrotomy to allow the insertion of respective jaws of a linear stapler. One jaw of the linear stapler is inserted in the gastrotomy made at the second tissue portion of the stomach. The other jaw of the linear stapler is inserted in the enterotomy made at the first tissue portion of the intestine. One flap of the two incisions is then sutured by the linear stapler by means of a sequence of points, joining the two tissue portions and partially defining the gastroenteroanastomosis.

In accordance with a preferred embodiment, the gastrotomy and enterotomy are realised before bringing the two tissue portions close together. In this case, the linear stapler is used as an instrument for bringing the two tissue portions close together, for example inserting one of the two jaws of the linear stapler into the enterotomy and using the stapler for bringing the first tissue portion close to the second tissue portion. The other jaw is then inserted into the gastrotomy so to join the two tissues.

To complete the anastomosis (gastroenteroanastomosis 16) between the two tissue portions, the flaps of the gastrotomy and enterotomy are reclosed, for example by means of suture. This allows restoring the continuity of the intestinal duct after the realisation of the gastroenteroanastomosis. Indeed, after having completed the gastroenteroanastomosis, the intestinal tract maintains its integrity and continuity, since the incisions carried out were restapled to form the gastroenteroanastomosis.

In accordance with a preferred embodiment, the step wherein the gastroenteroanastomosis 16 is completed is substantially realised at the end of the procedure, before sectioning the intestine and before carrying out the seal test, as will be described below.

Subsequently, an additional ansa of the intestine is chosen, distal with respect to the gastroenteroanastomosis with reference to the natural flow along the intestinal duct, i.e. to the flow before carrying out the gastrointestinal bypass. In other words, by distal it is intended an ansa downstream of the gastroenteroanastomosis 16 with reference to the natural flow along the intestinal duct. The definition of proximal or distal will also be used below with reference to the natural flow inside the intestinal duct.

A corresponding additional first tissue portion 18 of the additional intestine ansa, distal with respect to the gastroenteroanastomosis 16 with reference to the natural flow along the intestinal duct, is then brought close to an additional second tissue portion 20 of the intestine, proximal with respect to the natural flow along the intestinal duct, realising a second loop B of the intestine which is distal with respect to the gastroenteroanastomosis. In other words, as defined above, the additional first tissue portion 18 is arranged downstream of the gastroenteroanastomosis 16 with respect to the natural flow along the intestinal duct, moreover the additional second tissue portion 20 is arranged upstream of the gastroenteroanastomosis 16 with respect to the natural flow along the intestinal duct.

The additional second tissue portion 20 of the intestine, proximally arranged with respect to the gastroenteroanastomosis 16, is brought close to the gastroenteroanastomosis.

At each of the two additional tissue portions 18, 20, an incision (enterotomy) is made, adapted to receive a respective jaw of a linear stapler. A first jaw of the linear stapler is inserted in the enterotomy made at the first tissue portion of the intestine. A second jaw of the linear stapler is inserted in the enterotomy made at the additional second tissue portion of the intestine. A sequence of points is then applied, which partially unites the flaps of the enterotomy and partially defines the enteroenteroanastomosis.

In accordance with a preferred embodiment described above, the enterotomies are realised before bringing the two tissue portions close together. In this case, the linear stapler is used as an instrument for bringing the two tissue portions close together, for example inserting one of the two jaws of the linear stapler into the enterotomy made at the additional first distal tissue portion, and using the stapler for bringing the first distal tissue portion close to the additional second proximal tissue portion. The other jaw is then inserted in the enterotomy of the additional second proximal tissue portion so to join the two tissues.

The enteroenteroanastomosis 22 between the two tissue portions is subsequently completed by reclosing the flaps of the enterotomies so to restore the continuity of the intestinal duct after the realisation of the enteroenteroanastomosis. The remaining flaps of the enterotomies are joined for example by means of suture.

In accordance with a preferred embodiment, the step wherein the enteroenteroanastomosis 22 is completed is substantially realised at the end of the procedure, at the same time as the completion of the gastroenteroanastomosis before sectioning the intestine and before carrying out the seal test, as will be described below.

After having completed the enteroenteroanastomosis, the intestinal tract maintains its integrity and continuity, since the incisions carried out were restapled to form the enteroenteroanastomosis.

As illustrated in FIG. 2, the gastroenteroanastomosis and the enteroenteroanastomosis are rather close to each other, and permit operating in the upper part of the abdomen.

After having made the second loop, it is possible to carry out, preferably at the same time, a seal test of the two anastomoses, for example by means of methylene blue. The step of sectioning the intestine between the gastroenteroanastomosis and the enteroenteroanastomosis is realised at the end of the procedure, when the correct functioning of the two anastomoses was verified. In FIG. 2, it is indicated with a section line 24.

Advantageously, the aforesaid method is realised laparoscopically, comprising an initial step of inserting trocars, preferably four trocars arranged respectively in the following zones: epigastrium, left flank, and two trocars at the mesogastrium zone.

In accordance with the present invention, the instrumentation for carrying out a gastrointestinal bypass comprises:
means for bringing a first tissue portion 12 of the intestine close to a second tissue portion 14 of the stomach, realising a first loop A of the intestine between the stomach and the first tissue portion of the intestine,
means for creating a gastroenteroanastomosis 16 between the two portions of close tissue, maintaining or restoring the continuity of the intestinal duct after the realisation of the gastroenteroanastomosis,
means for bringing close together an additional first tissue portion 18 of the intestine which is distally arranged or downstream with respect to the gastroenteroanastomosis 16 with reference to the natural flow along the intestinal duct, and an additional second tissue portion 20 of the intestine arranged proximally, or upstream, with respect to the natural flow along the intestinal duct, realising a second loop B of the intestine which is distal with respect to the gastroenteroanastomosis 16, wherein said additional second tissue portion 20 of the intestine arranged proximally with respect to the gastroenteroanastomosis 16 is brought close to the gastroenteroanastomosis 16,
means for creating an enteroenteroanastomosis 22 between the two close tissue portions of the intestine, maintaining or restoring the continuity of the intestinal duct after the realisation of the enteroenteroanastomosis,
means for sectioning the intestine between the gastroenteroanastomosis 16 and the enteroenteroanastomosis 22.

Preferably, the aforesaid instrumentation moreover comprises means for carrying out a seal test both of the gastroenteroanastomosis 16 and the enteroenteroanastomosis 22, before the intestine is sectioned between the gastroenteroanastomosis 16 and the enteroenteroanastomosis 22. Still more preferably; the means for carrying out the seal test are adapted to simultaneously test both the gastroenteroanastomosis 16 and the enteroenteroanastomosis 22. Such means may comprise, for example, means for inserting and visualising methylene blue through the intestinal duct.

In accordance with a possible embodiment, the means for creating the gastroenteroanastomosis 16 comprise a linear stapler adapted to partially join the flaps of a gastrotomy and enterotomy respectively made at the first portion of the intestine and the second portion of the stomach. The means for creating the gastroenteroanastomosis 16 comprise moreover means for completing the gastroenteroanastomosis by reclosing the flaps still open after the use of the linear stapler, said means being adapted to restore the continuity of the intestinal duct after the realisation of the gastroenteroanastomosis. Preferably, the linear stapling also carries out the function of means for bringing close together the two tissue portions to be joined, inserting a jaw in the first tissue portion and using the stapler as a means for transporting the first tissue portion in correspondence with the second tissue portion.

In accordance with a possible embodiment, the means for creating the enteroenteroanastomosis comprise a linear stapler adapted to partially join the flaps of enterotomies respectively made at the additional first and second portions of the intestine. Moreover, the means for creating the enteroenteroanastomosis comprise means for completing the enteroenteroanastomosis by reclosing the flaps still open after the use of the linear stapler, said means being adapted to restore the continuity of the intestinal duct after the realisation of the enteroenteroanastomosis. Preferably, the linear stapling also carries out the function of means for bringing close together the two tissue portions to be joined, inserting a jaw in the first tissue portion and using the stapler as a means for transporting the first tissue portion in correspondence with the second tissue portion.

The instrumentation according to the present invention advantageously comprises means for preliminarily realising a gastric pouch, wherein the second tissue portion 14 of the stomach is arranged at the gastric pouch.

Preferably, the means used are adapted to realise the gastrointestinal bypass laparoscopically.

The method and the instrumentation described above may be applied both to the step of carrying out the gastroenteroanastomosis and the step of carrying out the enteroenteroanastomosis, or to one of these.

Such method and instrumentation allow reducing the risks of mortality with gastrointestinal bypasses and considerably limit the operation times. Maintaining the continuity of the intestine until the completion of the two anastomoses, it is possible to verify both simultaneously. Moreover, due to the close arrangement of the two anastomoses, the operation area is limited to the upper zone of the abdomen.

Additionally, the advantageous prevision of forming two intestine loops without previously interrupting the continuity allows choosing the correct length, so to avoid tensions. Moreover, the realisation of the gastroenteroanastomosis without preliminary sectioning of the ansae permits reducing the risk of incorrectly joining the segments or inducing undesired torsions.

Moreover, the combination of the present method results particularly innovative, by which the two intestine loops are made foreseeing the use of linear staplers for realising the anastomoses, preferably close to each other. In fact, beyond the above mentioned advantages, the use of the linear stapler permits limiting the size and extension of the bleeding, the losses and risk of stenosis. The use of the linear stapler for an application as previously described overcomes a deep-rooted disadvantage which previously prevented its application. In particular, the advantageous prevision of using a linear stapler for carrying out of both anastomoses in a method as described permits maintaining good blood perfusion of the affected tissues and permits having available an instrument of limited size adapted to operate in a restricted area of the abdomen.

In accordance with a different embodiment, the steps of bringing close together tissues and/or creating the gastroenteroanastomosis and/or enteroenteroanastomosis are realised intraluminally by using an anastomotic device as for example illustrated in FIGS. 3-6. Such device is preferably made to slide along a guide means, preliminarily inserted through the portions to be brought together and/or joined and making up part of the instrumentation according to the present invention. Preferably, the guide means comprises at least one guide cable 200 arranged as an open loop which crosses through the portions to be joined and which can be associated with an anastomotic device.

The partial realisation of a pouch 100 in the stomach may be previously foreseen, to which the first intestine portion will be connected. Subsequently, a first guide cable open loop C is realised through the open portion of the gastric pouch and through the first portion 12 of the intestine and the second portion 14 of the stomach to be joined. In accordance with one possible embodiment, the gastric pouch is realised at the end of the procedure, after the realisation of the gastroenteroanastomosis and the enteroenteroanastomosis. In such case, the first guide cable open loop C is realised through the stomach and the intestine, crossing the tissue portions to be united as for example illustrated in FIG. 3.

An anastomotic device is inserted and locked on the guide means, and transported, by means of the guide means itself, until it abuts against the first portion 12 to be joined and brings it close to the second portion 14 to be joined. This first sequence of steps concludes with the realisation of a gastroenteroanastomosis. The anastomotic device can be realised by means of an anvil adapted to be locked on the guide cable and adapted to cooperate with a circular stapler to carry out the gastroenteroanastomosis. Alternatively, the anastomotic device can be a device adapted to position an anastomotic ring, preferably elastic, to keep the two tissue portions joined (as for example illustrated in FIG. 5a with reference to the enteroenteroanastomosis) or other anastomotic devices adapted for such purpose.

Subsequently, a second guide cable open ring D is realised through the two portions of the intestine to be joined (additional first portion 18, distal with respect to the gastroenteroanastomosis, and additional second portion 20, proximal with respect to the gastroenteroanastomosis). If the gastric pouch is partially realised at the beginning of the procedure, the second guide cable open loop also crosses the open portion of the gastric pouch. FIG. 4 illustrates the second guide cable open loop D without preliminary, partial formation of the gastric pouch. The second guide cable open loop crosses the gastroenteroanastomosis 16, the additional first portion 18, distal with respect to the gastroenteroanastomosis 16 with reference to the natural flow of the intestine duct, the additional second portion 20, proximal with respect to the gastroenteroanastomosis, and has the two ends preferably at the same orifice. FIG. 7 illustrates a possible variation wherein the guide cable open loop D is realised by crossing the gastroenteroanastomosis 16, the additional first portion 18, distal with respect to the gastroenteroanastomosis 16 with reference to the natural flow of the intestine duct, the additional second portion 20, proximal with respect to the gastroenteroanastomosis, and has the two ends in correspondence preferably with the same orifice.

Also in this case, an anastomotic device is inserted and locked on the guide means, and transported, by means of the guide means itself, until it abuts against the additional first portion 18 to be joined and brings it close to the additional second portion 20 to be joined. This second sequence of steps concludes with the realisation of an enteroenteroanastomosis and the completion of the gastric pouch. As with the gastroenteroanastomosis, the anastomotic device can be realised by means of an anvil adapted to be locked on the guide cable and adapted to cooperate with a circular stapler to carry out the enteroenteroanastomosis. Alternatively, the anastomotic device can be a device adapted to position an anastomotic ring 30, preferably elastic, to keep the two tissue portions joined, or other anastomotic devices adapted for such purpose.

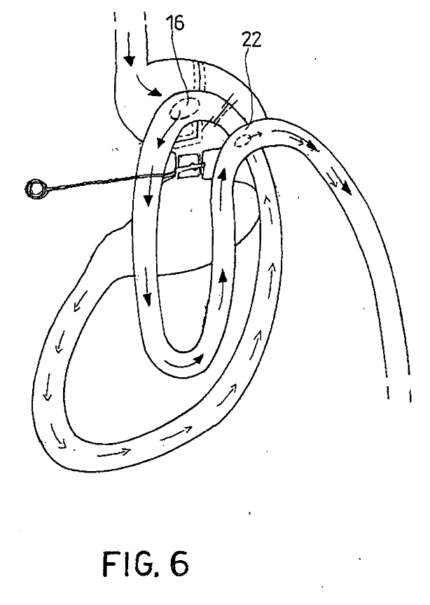

After having realised the gastroenteroanastomosis and the enteroenteroanastomosis, it is possible to simultaneously test both as previously described. Preferably, the step of the seal test is carried out after the realisation (or completion) of the gastric pouch. Finally, the intestine is sectioned between the gastroenteroanastomosis and the enteroenteroanastomosis with the section line 24. FIG. 5 illustrates this latter situation wherein also the realisation of the gastric pouch 100 is highlighted at the end of the procedure. FIG. 5a illustrates a detail of FIG. 5 wherein a possible formation is highlighted of the enteroenteroanastomosis by means of a preferably elastic loop released by an anastomotic positioner device. The same solution may possibly be adopted for the gastroenteroanastomosis 16. FIG. 6 illustrates a possible embodiment wherein a gastric bandage is realised.

The passage of the guide means through the walls of the tissues to be united can be realised by perforating the wall (for example with radiofrequency needles) at the zone intended to form the anastomosis, so that after the formation of the anastomosis the continuity of the intestinal duct is restored.

In accordance with a possible embodiment, the guide means and the anastomotic device as previously described may be used in any technique, for example hybrid intraluminal and laparoscopic or other type.

In accordance with a possible embodiment, the instrumentation according to the present invention foresees means for bringing close together the tissue portions comprising an anastomotic device adapted to bring close together and/or join the tissues intraluminally.

An anastomotic device adapted for such purpose can be a device adapted to release an anastomotic ring to realise the anastomosis, or a circular stapler sliding on the guide means and cooperating with an anvil, lockable on the guide means.

The instrumentation according to the present invention comprises means for partially realising a gastric pouch before inserting the guide means, wherein the second tissue portion 14 of the stomach is arranged at the gastric pouch, or means for realising the complete gastric pouch at the end of the procedure. In the first case, means are advantageously foreseen for completing the gastric pouch after the formation of the gastroenteroanastomosis and enteroenteroanastomosis.

The method and the instrumentation described above with reference to a procedure with guide means, preferably intraluminally, may be applied both to the step of carrying out the gastroenteroanastomosis and the step of carrying out the enteroenteroanastomosis, or to one of these.

As in the previous case, the foreseen method and instrumentation allow reducing the risks of mortality in gastrointestinal bypasses and considerably limiting the operation times. The maintenance of the continuity in the intestine until the completion of the two anastomosis permits the simultaneous verification of both. Moreover, due to the close arrangement of the two anastomoses, the operation area is limited to the upper zone of the abdomen.

A man skilled in the art, in order to satisfy specific and contingent needs, can make numerous modifications and adaptations to the preferred embodiments of the devices and methods described above, as well as substitute elements with other functionally equivalent ones, without however departing from the scope of the following claims.

1. Instrumentation for carrying out a gastrointestinal bypass comprising:
means for bringing a first tissue portion (12) of the intestine close to a second tissue portion (14) of the stomach, realising a first loop (A) of the intestine between the stomach and the first tissue portion,
means for creating a gastroenteroanastomosis (16) between the two close tissue portions, maintaining or restoring the continuity of the intestinal duct after the realisation of the gastroenteroanastomosis,
means for bringing close together an additional first tissue portion (18) of the intestine arranged distally or downstream with respect to the gastroenteroanastomosis (16) with reference to the natural flow along the intestinal duct, and an additional second tissue portion (20) of the intestine arranged proximally, or upstream, with respect to the gastroenteroanastomosis (16) with reference to the natural flow along the intestinal duct, realising a second loop (B) of the intestine which is distal with respect to the gastroenteroanastomosis (16), wherein said additional second tissue portion (20) of the intestine, proximal with respect to the gastroenteroanastomosis (16), is brought close to the gastroenteroanastomosis (16),
means for creating an enteroenteroanastomosis (22) between the two close tissue portions of the intestine, maintaining or restoring the continuity of the intestinal duct after the realisation of the enteroenteroanastomosis,
means for sectioning the intestine between the gastroenteroanastomosis (16) and the enteroenteroanastomosis (22).

2. Instrumentation according to claim 1, comprising means for carrying out a seal test both of the gastroenteroanastomosis (16) and the enteroenteroanastomosis (22), before the intestine is sectioned between the gastroenteroanastomosis (16) and the enteroenteroanastomosis (22).

3. Instrumentation according to claim 2, wherein the means for carrying out the seal test are adapted to simultaneously test both the gastroenteroanastomosis (16) and the enteroenteroanastomosis (22).

4. Instrumentation according to one of the preceding claims, wherein the means for creating the gastroenteroanastomosis (16) comprise a linear stapler adapted to partially join the flaps of a gastrotomy and enterotomy respectively made at the first portion of the intestine and the second portion of the stomach.

5. Instrumentation according to claim 4, wherein the means for creating the gastroenteroanastomosis (16) moreover comprise means for completing the gastroenteroanastomosis by reclosing the flaps still open after the use of the linear stapler, said means being adapted to restore the continuity of the intestinal duct after the realisation of the gastroenteroanastomosis.

6. Instrumentation according to one of the preceding claims, wherein the means for creating the enteroenteroanastomosis comprise a linear stapler adapted to partially join the flaps of enterotomies respectively made at the additional first and second portions of the intestine.

7. Instrumentation according to claim 6, wherein the means for creating the enteroenteroanastomosis moreover comprise means for completing the enteroenteroanastomosis by reclosing the flaps still open after the use of the linear stapler, said means being adapted to restore the continuity of the intestinal duct after the realisation of the enteroenteroanastomosis.

8. Instrumentation according to one of the claims from 4-7, comprising means for preliminarily making a gastric pouch, said second tissue portion (14) of the stomach being arranged at the gastric pouch.

9. Instrumentation according to one of the claims from 4-8, wherein the means used are adapted to realise the gastrointestinal bypass laparoscopically.

10. Instrumentation according to one of the claims 1-3, wherein the means for bringing the tissue portions close together comprise an anastomotic device adapted for bringing the tissues close together intraluminally.

11. Instrumentation according to one of the claims 1-3, wherein the means for creating the gastroenteroanastomosis (16) and/or enteroenteroanastomosis (22) comprise an anastomotic device adapted to realise the gastroenteroanastomosis (16) and/or enteroenteroanastomosis (22) intraluminally.

12. Instrumentation according to claim 10 or 11, comprising a guide means on which the anastomotic device is locked or made to slide, said guide means being preliminarily inserted through the tissue portions to be brought close together and/or joined, forming an open loop.

13. Instrumentation according to claim 12, comprising means for partially making a gastric pouch before inserting said guide means, said second tissue portion (14) of the stomach being arranged at the gastric pouch.

14. Instrumentation according to claim 13, comprising means for completing the gastric pouch after the formation of the gastroenteroanastomosis and enteroenteroanastomosis.

15. Instrumentation according to claim 12, comprising means for making a gastric pouch after the formation of the gastroenteroanastomosis (16) and the enteroenteroanastomosis (20), said second tissue portion (14) of the stomach being arranged at the gastric pouch.

16. Method for carrying out a gastrointestinal bypass comprising the steps of:
bringing a first tissue portion (12) of the intestine close to a second tissue portion (14) of the stomach, realising a first loop (A) of the intestine between the stomach and the first tissue portion,
creating a gastroenteroanastomosis (16) between the two close tissue portions, maintaining or restoring the continuity of the intestinal duct after the realisation of the gastroenteroanastomosis,
bringing close together an additional first tissue portion (18) of the intestine arranged distally or downstream with respect to the gastroenteroanastomosis (16) with reference to the natural flow along the intestinal duct, and an additional second tissue portion (20) of the intestine arranged proximally, or upstream, with respect to the gastroenteroanastomosis (16) with reference to the natural flow along the intestinal duct, realising a second loop (B) of the intestine which is distal with respect to the gastroenteroanastomosis (16), wherein said additional second tissue portion (20) of the intestine, proximal with respect to the gastroenteroanastomosis (16), is brought close to the gastroenteroanastomosis (16),
creating an enteroenteroanastomosis (22) between the two close tissue portions of the intestine, maintaining or restoring the continuity of the intestinal duct after the realisation of the enteroenteroanastomosis,
sectioning the intestine between the gastroenteroanastomosis (16) and the enteroenteroanastomosis (22).

17. Method according to claim 16, wherein the intestine is sectioned between the gastroenteroanastomosis (16) and the enteroenteroanastomosis (22) after a seal test step of both the gastroenteroanastomosis (16) and the enteroenteroanastomosis (22).

18. Method according to claim 17, wherein the seal test step occurs simultaneously for both the gastroenteroanastomosis (16) and the enteroenteroanastomosis (22).

19. Method according to one of the claims from 16-18, wherein the step of creating the gastroenteroanastomosis (16) is at least partially realised using a linear stapler.

20. Method according to claim 19, wherein one jaw of the linear stapler is inserted in a gastrotomy made at the second tissue portion of the stomach and the other jaw of the linear stapler is inserted in an enterotomy made at the first tissue portion of the intestine, to realise at least one sequence of points which partially defines the gastroenteroanastomosis.

21. Method according to claim 19 or 20, wherein said linear stapler is used for bringing close together the two tissue portions to be joined, inserting one jaw of the linear stapler in an enterotomy made at the first tissue portion (12) of the intestine and transporting, by means of said linear stapler, the first intestine portion (12) in correspondence with a gastrotomy made in the second portion (14) of the stomach, before joining the two tissue portions.

22. Method according to claim 20 or 21, wherein the gastroenteroanastomosis is completed by reclosing the flaps of the gastrotomy and enterotomy to restore the continuity of the intestinal duct after the realisation of the gastroenteroanastomosis.

23. Method according to claim 22, wherein the gastroenteroanastomosis is completed after having joined the additional first and second tissue portion (18, 20) to be joined.

24. Method according to one of the claims from 16-23, wherein the step of creating the enteroenteroanastomosis (22) is at least partially realised by using a linear stapler.

25. Method according to claim 24, wherein one jaw of the linear stapler is inserted in an enterotomy made at the additional first tissue portion of the intestine and the other jaw of the linear stapler is inserted in an enterotomy made at the additional second tissue portion of the intestine, to realise at least one sequence of points which partially defines the enteroenteroanastomosis.

26. Method according to claim 24 or 25, wherein said linear stapler is used for bringing close together the two tissue portions to be joined, inserting one jaw of the linear stapler in an enterotomy made at the additional first tissue portion (18) of the intestine and transporting, by means of said linear stapler, the additional first intestine portion (18) in correspondence with an enterotomy made in the additional second portion (20) of the intestine before joining the two tissue portions.

27. Method according to claim 25 or 26, wherein the enteroenteroanastomosis is completed by reclosing the flaps of the enterotomies to restore the continuity of the intestinal duct after the realisation of the enteroenteroanastomosis.

28. Method according to one of the claims from 19-27, wherein a preliminary step is foreseen for making a gastric pouch and wherein said second tissue portion (14) of the stomach is arranged at the gastric pouch.

29. Method according to one of the claims from 19-28, realised laparoscopically.

30. Method according to claim 29, comprising an initial insertion step of trocars in the upper left part of the abdomen, wherein one is inserted at the epigastrium, one at the left flank and two at the mesogastrium.

31. Method according to one of the claims 16-18, wherein the steps of bringing tissues together are realised intraluminally by using an anastomotic device.

32. Method according to one of the claims 16-18, wherein the steps of creating the gastroenteroanastomosis (16) and/or enteroenteroanastomosis (22) are realised intraluminally by using an anastomotic device.

33. Method according to claim 31 or 32, wherein the anastomotic device is made to slide or is locked along a guide means preliminarily inserted through the tissue portions to be brought close together and/or joined, and shaped as an open loop.

34. Method according to claim 33, wherein the guide means is inserted along the intestine through the first tissue portion (12) of the intestine and through the second tissue portion (14) of the stomach, and shaped to form the first open loop (C) for bringing the first and second tissue portion close together and/or for realising the gastroenteroanastomosis (16).

35. Method according to claim 34, wherein the guide means is subsequently inserted through the gastroenteroanastomosis (16), the additional first portion (18), distal with respect to the gastroenteroanastomosis (16) with reference to the natural flow in the intestinal duct, the additional second portion (20), proximal with respect to the gastroenteroanastomosis (16) with reference to the natural flow in the intestinal duct, to form a second open loop (D) for bringing the additional first and second tissue portions close together and/or realising the enteroenteroanastomosis (22).

36. Method according to claim 34, wherein the guide means is subsequently inserted through the gastroenteroanastomosis (16), the additional first portion (18), distal with respect to the gastroenteroanastomosis (16) with reference to the natural flow in the intestinal duct, the additional second portion (20), proximal with respect to the gastroenteroanastomosis (16) with reference to the natural flow in the intestinal duct, again the gastroenteroanastomosis (16), to form a second open loop (D) for bringing the additional first and second tissue portions close together and/or realising the enteroenteroanastomosis (22).

37. Method according to one of the claims from 33-36, wherein a preliminary step is foreseen for partially making a gastric pouch, carried out before inserting said guide means, and wherein said second tissue portion (14) of the stomach is arranged at the gastric pouch.

38. Method according to claim 37, wherein said gastric pouch is completed after the formation of the gastroenteroanastomosis and enteroenteroanastomosis.

39. Method according to one of the claims 33-36, wherein a realisation step is foreseen of a gastric pouch (100), carried out after the realisation of the gastroenteroanastomosis and enteroenteroanastomosis.

40. Method according to one of the claims 16-18, wherein the steps of bringing tissues close together are realised by using an anastomotic device which is made to slide or is locked along a guide means preliminarily inserted through the tissue portions to be brought close together and/or joined, and shaped as an open loop.

41. Method according to one of the claims 16-18, wherein the steps of creating the gastroenteroanastomosis (16) and/or the enteroenteroanastomosis (22) are realised by using an anastomotic device which is made to slide or is locked along a guide means preliminarily inserted through the tissue portions to be brought close together and/or joined, shaped as an open loop.

42. Method according to claim 40 or 41, wherein the guide means is inserted along the intestine through the first tissue portion (12) of the intestine and through the second tissue portion (14) of the stomach, and shaped to form a first open loop (C) for bringing the first and second tissue portion close together and/or for realising the gastroenteroanastomosis (16).

43. Method according to claim 42, wherein the guide means is subsequently inserted through the gastroenteroanastomosis (16), the additional first portion (18), distal with respect to the gastroenteroanastomosis (16) with reference to the natural flow in the intestinal duct, the additional second portion (20), proximal with respect to the gastroenteroanastomosis (16) with reference to the natural flow in the intestinal duct, to form a second open loop (D) for bringing the additional first and second tissue portions close together and/or realising the enteroenteroanastomosis (22).

44. Method according to claim 42, wherein the guide means is subsequently inserted through the gastroenteroanastomosis (16), the additional first portion (18), distal with respect to the gastroenteroanastomosis (16) with reference to the natural flow in the intestinal duct, the additional second portion (20), proximal with respect to the gastroenteroanastomosis (16) with reference to the natural flow in the intestinal duct, again the gastroenteroanastomosis (16), to form a second open loop (D) for bringing the additional first and second tissue portions close together and/or realising the enteroenteroanastomosis (22).

45. Method according to one of the claims 40-44, wherein the steps of bringing the tissues close together and/or creating the gastroenteroanastomosis (16) and/or the enteroenteroanastomosis (22) are at least partially realised intraluminally.

46. Method according to claim 45, wherein a preliminary step is foreseen for partially making a gastric pouch, carried out before inserting said guide means, and wherein said second tissue portion (14) of the stomach is arranged at the gastric pouch.

47. Method according to claim 46, wherein said gastric pouch is completed after the formation of the gastroenteroanastomosis and enteroenteroanastomosis.

48. Method according to claim 45, wherein a realisation step is foreseen of a gastric pouch (100), carried out after the realisation of the gastroenteroanastomosis and enteroenteroanastomosis.
